(19) United States
(12) Patent Application Publication
(10) Pub. No.: US 2026/0144472 A1
(43) Pub. Date: May 28, 2026

(54) PATCH DEVICE FOR CARDIAC ELECTRICAL ACTIVITY MONITORING

(71) Applicant: Everbeat, Inc., Rockville, MD (US)

(72) Inventor: George Stuart MENDENHALL, Del Mar, CA (US)

(73) Assignee: Everbeat, Inc., Rockville, MD (US)

(21) Appl. No.: 19/400,813

(22) Filed: Nov. 25, 2025

Related U.S. Application Data
(60) Provisional application No. 63/725,437, filed on Nov. 26, 2024.

(51) Int. Cl. A61B 5/28; A61B 5/00; A61B 5/113; A61B 5/1455; A61B 5/256; A61B 5/259

(52) U.S. Cl. CPC A61B 5/28; A61B 5/1135; A61B 5/14552; A61B 5/256; A61B 5/259; A61B 5/4815; A61B 5/6826; A61B 2560/0219; A61B 2562/0219

(57) ABSTRACT

A patch device for monitoring cardiac electrical activity is provided. The patch device comprises a flexible substrate that is configured to adhere to skin of a user and that comprises two or more electrodes. The patch device further comprises a port for releasably attaching a ring device, wherein the ring device is configured to be worn around a finger of the user and comprises a monitor configured to monitor cardiac electrical activity of the user, wherein the port is configured to electrically couple the two or more electrodes of the flexible substrate to the monitor of the ring device.

### CROSS-REFERENCE TO RELATED APPLICATIONS

This application claims the benefit of U.S. Provisional Application No. 63/725,437, filed Nov. 26, 2024, the entire contents of which is incorporated herein by reference.

### FIELD

The present disclosure relates generally to wearable devices for cardiac monitoring, and more specifically to patch devices for generating electrocardiography data.

### BACKGROUND

Cardiac monitoring is essential for the detection of life-threatening cardiovascular diseases. Traditional diagnostic methods, such as electrocardiography, typically require the use of multiple electrodes and complex setups in healthcare facilities such as hospitals and/or doctors'offices, limiting their widespread use. Such approaches may also fail to capture sporadic cardiac events such as intermittent arrhythmia due to their brief monitoring duration and lack of continuous data collection.

Wearable devices may address some of these challenges by enabling cardiac monitoring outside of clinical settings. However, many wearable devices may be limited in their ability to provide continuous and reliable cardiac monitoring and may involve a user remaining in contact with a particular point on a device. This limitation may reduce the ability of such devices to capture sporadic events or to perform uninterrupted monitoring. Additionally, known wearable devices that do enable continuous monitoring may be designed for single-use or limited wear time. Such devices may be dedicated to electrocardiography measurements or may include a separate device to integrate additional readings, for example a finger clip pulse oximeter to detect sleep apnea events. The use of separate devices may introduce additional challenges, including user discomfort, increased system complexity, and potential signal loss or degradation if the device becomes dislodged.

### SUMMARY

Disclosed herein are examples of devices and methods for physiological monitoring of a user that integrate a ring device configured for measuring cardiac activity with an adherent patch device that enables the ring device to be used for continuous monitoring of cardiac activity. The ring device may include two or more electrodes configured for monitoring cardiac electrical activity of a user when worn on the user's finger. An exemplary patch device may include a port for releasably attaching and interfacing to the ring device, thereby enabling the cardiac monitoring capabilities of the ring device to be used in similar fashion to a patch-type cardiac monitor. For example, connectors of the port may electrically couple the two or more electrodes of the ring device with two or more electrodes of a flexible substrate that forms the patch device. When an exemplary patch device is adhered to the skin of the user and the ring is releasably attached to the port, electrodes of the ring device may couple to electrodes of the flexible substrate in contact with the user's skin. This coupling may allow the ring device to measure the electric potential across the two electrodes of the flexible substate, continuously generating cardiac activity data.

Such continuous monitoring may persist during all of a user's activities, including sleep, daily routines, and/or periods of physical activity, such as sports or exercise.

The port may include a light pipe to optically couple an LED and a photodiode of a pulse oximetry sensor of the ring device with the skin of the user, thereby allowing the ring device to continuously measure parameters such as the blood oxygen saturation and/or pulse rate of a user. The ring device may include an accelerometer for detecting and/or characterizing user motion. The user's respiratory motion may be monitored based on signals detected by the accelerometer of the ring device. Monitoring of cardiac activity, blood oxygen saturation, pulse rate, and/or respiratory motion by an exemplary patch device may enable monitoring of the respiratory activity of a user overnight and may enable detection of, for example, a sleep apnea event, and notification of the user via an alarm.

An exemplary patch device may include an auxiliary battery and may be configured to power a ring device attached to the port of the patch device. In this way, the patch device may enable continuous recording of electrocardiography, pulse oximetry, and/or accelerometer data for an extended period of time without requiring external charging.

In some embodiments, a patch device for monitoring cardiac electrical activity is provided, the patch device comprising a flexible substrate configured to adhere to skin of a user and comprising two or more electrodes; and a port for releasably attaching a ring device, wherein the ring device is configured to be worn around a finger of the user and comprises a monitor configured to monitor cardiac electrical activity of the user, wherein the port is configured to electrically couple the two or more electrodes of the flexible substrate to the monitor of the ring device.

In some embodiments, the two or more electrodes of the flexible substrate comprise electrically conductive adhesive gel configured to adhere to the skin of the user. In some embodiments, the at least one cardiac electrical activity signal is based on an electric potential between the two or more electrodes of the flexible substrate. In some embodiments, the ring device comprises two or more electrodes configured to detect cardiac electrical activity when contacting the skin of the user. In some embodiments, the port is configured electrically couple at least one of the two or more electrodes of the flexible substrate to at least one electrode of the two or more electrodes of the ring device when the ring device is releasably attached to the port. In some embodiments, the port comprises at least one inner connector, at least one outer connector, or both; and at least one connector, selected from a group consisting of the at least one inner connector and the at least one outer connector, comprises a tapered interface, a spring-loaded interface, or a tapered spring-loaded interface. In some embodiments, the port comprises at least one inner connector configured to electrically couple at least one electrode located at an inward-facing surface of the ring device to a first electrode of the two or more electrodes of the flexible substrate; and at least one outer connector configured to electrically couple at least one electrode located at an outward-facing surface of the ring device to a second electrode of the two or more electrodes of the flexible substrate. In some embodiments, the port comprises a first inner connector configured to electrically couple a first electrode located at an inward-facing surface of the ring device to a first electrode of the two or more electrodes of the flexible substrate; and a second inner connector configured to electrically couple a second electrode located at an inward-facing surface of the ring device to a second electrode of the two or more electrodes of the flexible substrate. In some embodiments, the port comprises a first outer connector configured to electrically couple a first electrode located at an outward-facing surface of the ring device to a first electrode of the two or more electrodes of the flexible substrate; and a second outer connector configured to electrically couple a second electrode located at an outer-facing surface of the ring device to a second electrode of the two or more electrodes of the flexible substrate. In some embodiments, the patch device further comprises a light pipe, wherein the light pipe is configured to direct light between the skin of a user and a pulse oximetry sensor of the ring device. In some embodiments, the patch device further comprises a pulse oximetry sensor configured to measure a blood oxygen saturation of the user, a pulse rate of the user, or both, wherein the pulse oximetry sensor comprises an LED and a photodetector. In some embodiments, the patch device further comprises a transceiver configured to transmit data from the pulse oximetry sensor to a transceiver of the ring device. In some embodiments, the patch device further comprises a battery for charging the ring device. In some embodiments, the patch device is configured to inductively charge the ring device.

In some embodiments, an apparatus for monitoring cardiac electrical activity is provided, the apparatus comprising a ring device configured to be worn around a finger of a user and comprising a monitor configured to monitor the cardiac electrical activity of the user; a flexible substrate configured to adhere to skin of the user and comprising two or more electrodes; and a port configured to releasably attach the ring device and electrically couple the two or more electrodes of the flexible substrate to the monitor of the ring device.

In some embodiments, a method for monitoring a user is provided, the method comprising adhering, to skin of a user, a patch device configured to releasably attach a ring device; measuring a cardiac electrical activity signal of the user based on an electric potential between two or more electrodes of the ring device when the patch device is adhered to the skin of the user and the ring device is releasably attached to the patch device; and generating data to simulate an electrocardiogram based on the measured cardiac electrical activity signal of the user.

In some embodiments, a method for monitoring a user is provided, the method comprising adhering, to skin of a user, a patch device configured to releasably attach a ring device; and measuring a blood oxygen saturation of the user, a pulse rate of the user, or both, based on a signal from a pulse oximetry sensor when the patch device is adhered to the skin of the user and the ring device is releasably attached to the patch device.

In some embodiments, a method for monitoring a user is provided, the method comprising adhering, to skin of a user, a patch device configured to releasably attach a ring device; and measuring a signal from an accelerometer of the ring device when the patch device is adhered to the skin of the user and the ring device is releasably attached to the patch device.

In some embodiments, the method further comprises detecting, in response to the signal from the accelerometer, a body position of the user and a respiratory movement of the user; determining, based on the respiratory movement of the user, that a respiratory activity of the user has diminished; and generating, based on the body position of the user and the determination that the respiratory activity of the user has diminished, an alarm signal. In some embodiments, the alarm signal is generated based on a detection of at least one body position of the user selected from a group comprising: lying on the back, lying on the right side, and lying on the left side. In some embodiments, the method further comprises detecting, in response to the signal from the accelerometer, a movement of the user; determining, based on the movement of the user, one or more patterns of body acceleration of the user; and computing, based on the one or more patterns of body acceleration of the user, at least one of: a number of steps taken by the user, a distance traveled by the user, or an amount of calories burned by the user. In some embodiments, the method further comprises detecting, in response to the signal from the accelerometer, and a movement of the user; determining, based on the movement of the user, one or more patterns of body acceleration of the user; and computing, based on the one or more patterns of body acceleration of the user, a postural stability of the user, a gait stability of the user, or both. In some embodiments, the method further comprises

detecting, in response to the signal from the accelerometer, a movement of the user;

determining, based on the movement of the user, that the user has suffered a fall; and

generating, based on the determination that the user has suffered a fall, an emergency notification.

In some embodiments, a method for monitoring a user is provided, the method comprising adhering, to skin of a user, a patch device configured to releasably attach a ring device; and measuring a signal from an accelerometer of the ring device and a signal from a pulse oximetry sensor when the patch device is adhered to the skin of the user and the ring device is releasably attached to the patch device.

In some embodiments, the method further comprises detecting, in response to the signal from the accelerometer, a body position of the user and a respiratory movement of the user; detecting, in response to the signal from the pulse oximetry sensor, a blood oxygen saturation of the user; determining, based on the respiratory movement of the user and the blood oxygen saturation of the user, that a respiratory activity of the user has diminished; and

generating, based on the body position of the user and the determination that the respiratory activity of the user has diminished, an alarm signal. In some embodiments, the alarm signal is generated based on a detection of at least one body position of the user selected from a group comprising: lying on the back, lying on the right side, and lying on the left side. In some embodiments, the method further comprises detecting, in response to the signal from the accelerometer, a body position of the user and a movement of the user; detecting, in response to the signal from the pulse oximetry sensor, a blood oxygen saturation of the user and a pulse rate of the user; determining, based on at least one of the body position of the user, the movement of the user, the blood oxygen saturation of the user, or the pulse rate of the user, one or more physiological patterns of the user; and generating an indication of sleep quality based on the one or more determined physiological patterns of the user.

In some embodiments, a non-transitory computer readable storage medium storing instructions for monitoring a user is provided, wherein the instructions, when executed by one or more processors of an electronic device, cause the device to measure a cardiac electrical activity signal of the user based on an electric potential between two or more electrodes of a ring device when a patch device is adhered to skin of the user and the ring device is releasably attached to the patch device; and generate data to simulate an electrocardiogram based on the measured cardiac electrical activity signal of the user.

In some embodiments, a non-transitory computer readable storage medium storing instructions for monitoring a user is provided, wherein the instructions, when executed by one or more processors of an electronic device, cause the device to measure a blood oxygen saturation of the user, a pulse rate of the user, or both, based on a signal from a pulse oximetry sensor when a patch device is adhered to skin of the user and a ring device is releasably attached to the patch device.

In some embodiments, a non-transitory computer readable storage medium storing instructions for monitoring a user is provided, wherein the instructions, when executed by one or more processors of an electronic device, cause the device to measure a signal from an accelerometer of a ring device when a patch device is adhered to skin of the user and the ring device is releasably attached to the patch device.

In some embodiments, a non-transitory computer readable storage medium storing instructions for monitoring a user is provided, wherein the instructions, when executed by one or more processors of an electronic device, cause the device to measure a signal from an accelerometer of a ring device and a signal from a pulse oximetry sensor when a patch device is adhered to skin of the user and the ring device is releasably attached to the patch device.

In some embodiments, any of the features of any of the embodiments described above and/or described elsewhere herein may be combined, in whole or in part, with one another. Additional advantages will be readily apparent to those skilled in the art from the following figures and detailed description. The aspects and descriptions herein are to be regarded as illustrative in nature and not restrictive.

### BRIEF DESCRIPTION OF THE FIGURES

A better understanding of the features and advantages of the present disclosure will be obtained by reference to the following detailed description that sets forth illustrative embodiments, in which the principles of the disclosure are utilized, and the accompanying figures of which:

FIG. 1 depicts an exemplary patch device adhered to the chest of a user, according to some embodiments.

FIG. 2A depicts a top view of an exemplary patch device without a ring device releasably attached, according to some embodiments.

FIG. 2B depicts a top view of an exemplary patch device with a ring device releasably attached, according to some embodiments.

FIG. 2C depicts a detail view of a port of an exemplary patch device, according to some embodiments.

FIG. 2D depicts a side view of an exemplary patch device with a ring device releasably attached, according to some embodiments.

FIG. 2E depicts a bottom view of an exemplary patch device, according to some embodiments.

FIG. 2F depicts a top view of an exemplary patch device including wiring traces, according to some embodiments.

FIG. 3 depicts an exemplary block diagram of components of a ring device and a patch device, according to some embodiments.

FIG. 4A depicts an exemplary process for using electrodes to measure cardiac electrical activity, according to some embodiments.

FIG. 4B depicts an exemplary process for using a pulse oximetry sensor to measure blood oxygen saturation and/or pulse rate, according to some embodiments.

FIG. 4C depicts an exemplary process for using an accelerometer to determine a diminished respiratory activity of a user, an exercise quality of a user, a postural stability and/or gait stability of a user, and/or that a user has suffered a fall, according to some embodiments.

FIG. 4D depicts an exemplary process for using an accelerometer and/or a pulse oximetry sensor to determine a diminished respiratory activity of a user and/or a sleep quality of a user, according to some embodiments.

FIG. 5 depicts an exemplary computing system, according to some embodiments.

### DETAILED DESCRIPTION

Disclosed herein are devices and methods for monitoring cardiac electrical activity that integrate a patch device configured to be adhered to a user's chest with a detachable ring device. The ring device may include a plurality of electrodes that enable the ring device to monitor cardiac electrical activity when worn on a finger. The patch device may enable the ring device to be used as a patch-type cardiac monitor, thereby enabling continuous cardiac electrical activity monitoring. The integrated patch and ring device can be placed on a user's chest for continuous monitoring of cardiac electrical activity. After a desired period of continuous monitoring is complete, the ring device can be detached from the patch device and can be worn on a user's finger for periodic electrical activity monitoring.

The patch device may serve as a stable interface for the ring device, providing electrical, mechanical, and/or optical coupling to ensure accurate data acquisition across a variety of user activities and/or environmental conditions. This integration may enable the ring device to make continuous measurements of cardiac electrical activity, blood oxygen saturation, pulse rate, and/or motion-related metrics, enabling the detection of critical health events such as arrhythmias, sleep apnea, near or full syncope (loss of consciousness) and/or falls over extended periods. In addition, by generating continuous electrocardiography data, the patch device may enable detection of cardiac pauses that may prompt an indication for a permanent pacemaker. Additionally, by generating continuous electrocardiography data, the patch device may enable identification of other arrhythmias, such as ventricular tachycardia, supraventricular tachycardia, and/or atrial fibrillation, which may result in treatment via anticoagulation (e.g. blood thinner use), antiarrhythmic therapy, and/or insertion of an implantable cardioverter-defibrillator.

The patch device may include a flexible substrate and at least two electrodes integrated with the flexible substrate. One or more of the at least two electrodes may include a dry-contact electrode for extended wear with minimal risk of skin irritation and/or may include gel and/or liquid for reduced impedance and/or improved skin contact. The flexible substrate may include multiple layers, such as for example, an elastomeric base for skin conformity, an adhesive layer for secure attachment, and/or an insulating dielectric layer to protect internal components. Embedded within the flexible substrate may be conductive traces that electrically couple to the electrodes. The patch device may include a port to releasably attach the ring device to the patch device. The port may include electrical connectors that are electrically coupled to the traces of the substrate and that are configured to mechanically and/or electrically couple to electrodes of the ring device when the ring device is mounted to the port, thereby connecting the electrodes of the flexible substrate to electrodes of the ring device. The port may include one or more tapered and/or spring-loaded interfaces (which may be provided by the electrical connectors) to securely but removable mount the ring device. The interface(s) may be configured accommodate variations in ring device sizes.

The ring device may include a pulse oximetry sensor and the patch device may include a light pipe configured to optically couple the pulse oximetry sensor to the user's skin. The light pipe may transmit light from an LED of the sensor to the user's skin and return light from the user's skin to a photodetector of the sensor. The patch device may further include a battery and circuitry (e.g., an inductive charger) for delivering charge to the ring device to extend the operating time of the ring device. The ring device and/or patch device may include one or more movement sensors, such as one or more accelerometer, gyroscopic sensors, and/or magnetometer sensors, for monitoring movement of a user.

The ring device may generate and store data from various sensors including the electrodes, the pulse oximetry sensor, and/or a movement sensor using an on-board controller and memory. The ring device may transmit data, for example using Bluetooth radiofrequency transmission, via an on-board transceiver to an external computing device, such as a mobile phone, for further analysis and/or storage.

Data collected by the ring device while attached to the patch device may be analyzed by the ring device and/or by an external computing device (e.g., a user's smartphone, a server system, etc.) to generate various insights and/or user alerts. For example, the ring device may generate simulated electrocardiograms during continuous cardiac monitoring, track respiratory activity to detect and/or notify users of sleep apnea events, and/or analyze motion patterns to assess activity levels, gait stability, and/or fall incidents. Electrocardiogram data may be provided to a physician for review on a real-time and/or continuous basis, and/or may be provided after-the-fact for non-synchronous review. This review and/or analysis may provide critical diagnostic information, such as identifying cardiac pauses that may occur while a user is sleeping and that may involve resolution via implantation of a pacemaker. Such information may result in the detection of atrial fibrillation that may result in the use of anticoagulation therapy (e.g., blood thinners) to reduce stroke risk. In the case of real-time review, such analysis may enable healthcare providers to act promptly based on detected abnormalities. The integration of accelerometer and pulse oximetry signals may support advanced analyses, such as evaluating sleep quality based on body position, movement patterns, blood oxygen levels, and/or heart rate variability.

The auxiliary battery of the patch device may extend the amount of time the ring device can operate without requiring recharging, enabling continuous monitoring of physiological signals by the ring device for an extended period of time. For example, the ring device may continuously monitor physiological signals for at least two weeks. The patch device may be water resistant and configured to operate under various environmental conditions, including sweat and motion during exercise and/or exposure to water. For example, adhesives used to attach the patch device to a user's skin may be configured to enable secure attachment even in high-moisture environments. The components of the patch device may include protective coatings and/or may be housed in casings to maintain functionality and/or to improve durability.

The patch device and ring device may be packaged and/or provided as a single kit. This kit may include the reusable ring device, one or more patch devices, and/or instructions for use. Such a kit may thus simplify the steps a user may undertake to begin monitoring electrocardiography and/or pulse oximetry data. As discussed above, after a period of continuous monitoring, a ring device may be detached from the patch device and worn on a user's finger for periodic cardiac electrical activity monitoring. An exemplary kit including a patch device and a ring device may thus provide continued usability beyond an initial monitoring period, distinguishing it from known wearable monitoring devices that may be discarded after a monitoring period that may last, for example, two weeks.

Users may extend the monitoring period by purchasing new patches separately, allowing the same ring device to be reused. For example, if a patch device detaches during use, the user may simply replace it with a new patch and continue using the existing ring device without disruption. Additionally, if a user would like to continue the monitoring period or conduct a second continuous monitoring period at a later date, the used patch device may simply be replaced with a new patch device. Thus, by separating elements that may be reused from elements that may be disposed of following use, an exemplary patch and ring device kit may offer advantages in usability and/or cost. As an alternative to continuous, day-and-night monitoring, a user may choose to wear the ring device for daytime cardiac electrical activity monitoring, and may attach the ring device to a patch device at night to continuously monitor cardiac electrical activity, blood oxygen saturation, pulse rate, and/or respiratory movements, thereby detecting arrhythmias and/or sleep apnea events while generating insights into the user's sleep quality. Such nighttime monitoring may additionally allow charging of the battery or energy storage device on-board the ring device, thereby enabling subsequent daytime monitoring.

In the following description of the various embodiments, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed terms. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some embodiments also relates to a device for performing the operations herein. This device may be specially constructed for the required purposes, or it may comprise a general-purpose computer selectively activated or reconfigured by a computer program stored in the computer. Such a computer program may be stored in a non-transitory, storage medium, such as, but not limited to, any type of disk, including floppy disks, USB flash drives, external hard drives, optical disks, CD-ROMs, magneto-optical disks, read-only memories (ROMs), random access memories (RAMs), EPROMs, EEPROMs, magnetic or optical cards, application-specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The structure for a variety of these systems will appear in the description below. In addition, the present invention is not described with reference to any particular programming language. It will be appreciated that a variety of programming languages may be used to implement the teachings of the present disclosure as described herein.

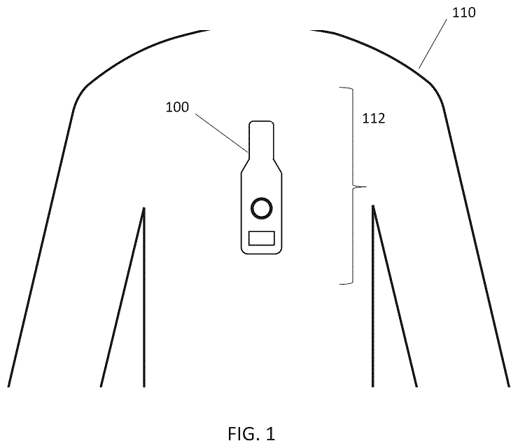

FIG. 1 depicts a user 110 with an exemplary patch device 100 adhered to their skin. In some implementations, user 110 may adhere patch device 100 to their chest region 112. For example, patch device 100 may be adhered at approximately the center of a user's chest region 112 and/or oriented vertically. As described below, patch device 100 may be adhered to the user's skin via adhesive. In some implementations, such adhesive may form part of electrodes of patch device 100 between which a ring device releasably attached to patch device 100 may measure electric potential. Additionally or alternatively, the adhesive may form part of a flexible substrate that forms patch device 100. By adhering patch device 100 to approximately the center of a user's chest region 112, electrodes of patch device 100 may be located on either side of the heart enabling a cardiac electrical activity signal to be measured based on the electric potential between the two electrodes when a ring device is releasably attached to patch device 100.

Such a ring device may be configured to be worn around the finger of a user and may include two or more electrodes configured to detect cardiac electrical activity when contacting the skin of the user, for example the skin of the user's finger. For example, the ring device may include a monitor, for example an integrated continuous monitor, configured to monitor cardiac electrical activity of the user. As described above, patch device 100 may be configured to couple the two or more electrodes of the patch device to the electrodes of the ring device, thereby coupling the electrodes of the patch device to the monitor of the ring device. The monitor of the ring device may be configured to simulate a continuous electrocardiogram based on the cardiac electrical activity signal measured using the electrodes of the patch device, as described in more detail below.

By adhering patch device 100 to approximately the center of their chest region 112, user 110 may attach patch device 100 to a stable and/or flat surface of the user's skin, thereby improving the accuracy and reliability of additional signals measured by the ring device. For example, the ring device may include a movement sensor, for example a three-axis accelerometer, and/or a pulse oximetry sensor, and a controller on the ring device may be configured to monitor the motion of the user's body using a signal from the accelerometer, and/or to monitor, for example, a blood oxygen saturation of level of user 110 using a signal from the pulse oximetry sensor. By adhering patch device 100 to approximately the center of their chest region 112, above their sternum bone, the user 110 may ensure the patch device is less susceptible to motion that may be experienced only by isolated portions of the user's body, for example motion of the user's pectoral muscles and/or breasts.

Thus, the approximate center of the user's chest region 112 may represent a comfortable and stable position to which to adhere patch device 100, enabling patch device 100 to be worn continuously for several days and/or weeks at a time, allowing detection and treatment of health events such as arrhythmias and/or sleep apnea episodes that may have otherwise gone unnoticed. Additionally or alternatively, to conserve battery life, patch device 100 may be configured to enable periodic measurement of physiological signals, for example measurement for a specified duration every hour and/or measurement triggered by detection of a user's motion and/or body position. For example, a detection of a sleep apnea event by one sensor may trigger additional monitoring. Similarly, with repeated findings of normal pulse rate and/or blood oxygen saturation level, monitoring may be decreased in frequency to enable an energy-efficient baseline screening.

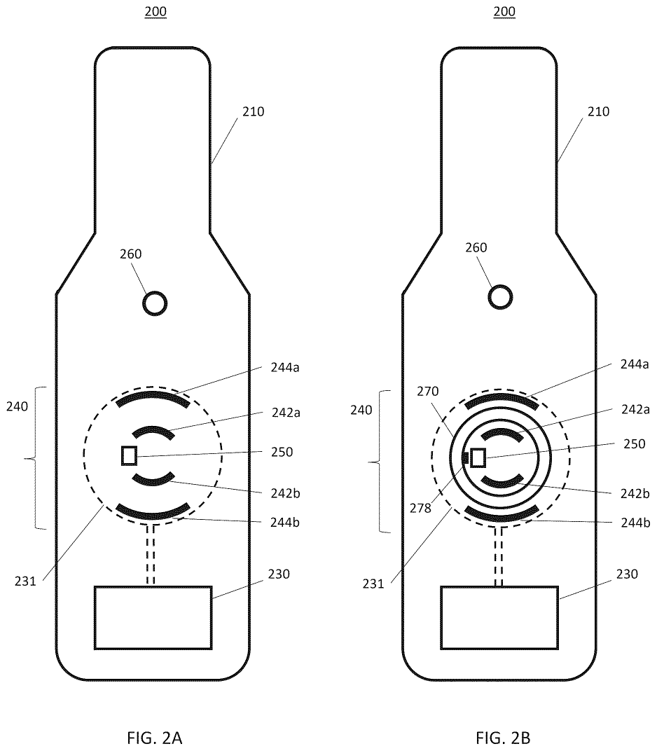

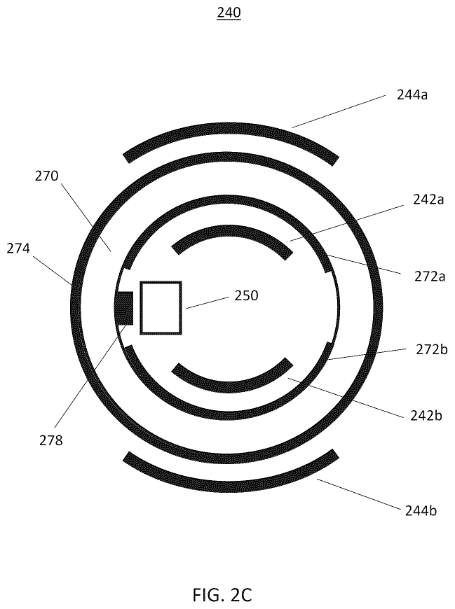

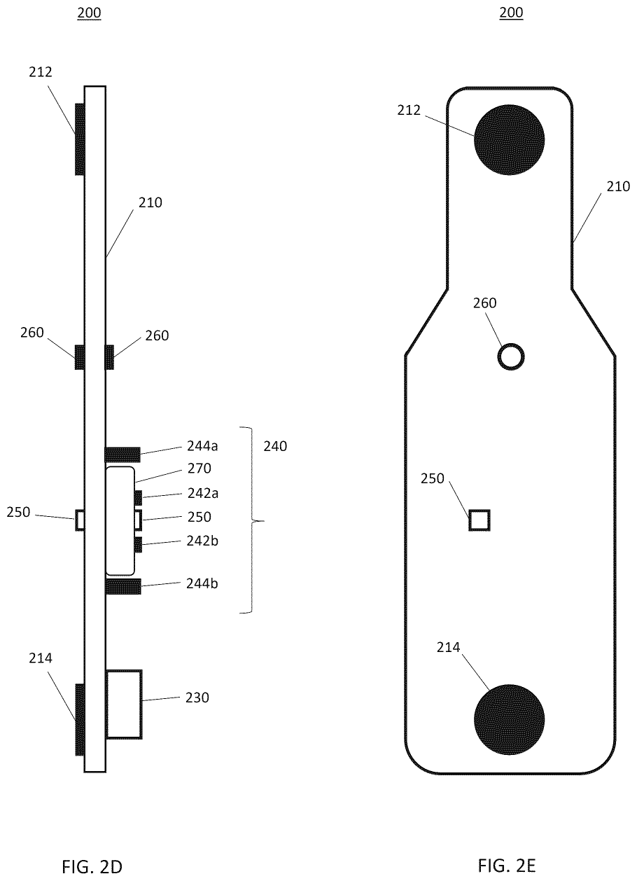

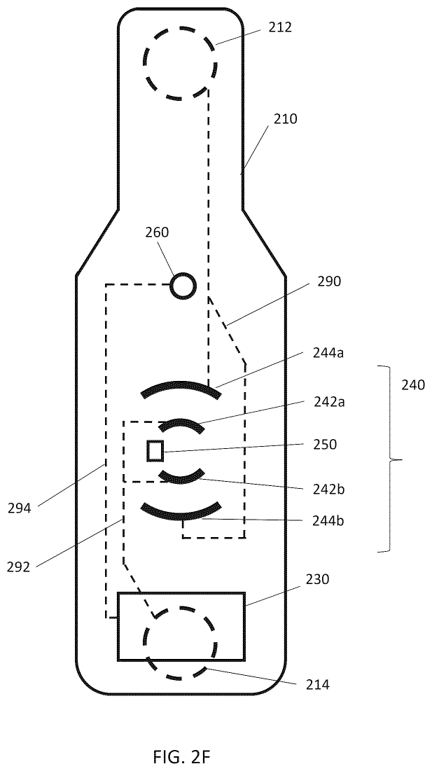

FIG. 2A depicts a top view of an exemplary patch device 200 without a ring device releasably attached. Patch device 200 may include a flexible substrate 210, a port 240 for releasably attaching a ring device, a battery 230 for charging a ring device, and/or a dedicated pulse oximetry sensor 260. Flexible substrate 210 may include an inductive charger (e.g., including an inductive charging coil 231) that may inductively charge a ring device when it is releasably attached to port 240. Flexible substrate 210 may include a multilayer design; for example, an elastomeric base layer to conform to a user's skin, an adhesive layer for secure attachment to the user's skin, and/or an insulating dielectric layer to electrically isolate electrical connections.

Additionally or alternatively, a wiring layer may be embedded within flexible substrate 210 and may include one or more conductive traces made from materials such as copper and/or silver/silver chloride, as depicted in FIG. 2F. Such traces may connect electrodes of the flexible substrate to port 240, facilitating electrical coupling to a ring device when releasably attached to the patch device. Traces within the wiring layer may additionally or alternatively connect battery 230 to the inductive charger (e.g., including an inductive charging coil 231) charging a ring device when attached and/or to the dedicated pulse oximetry sensor 260. To increase the durability of the wiring later, the layer may be encapsulated within the above-mentioned elastomeric base layer that may include, for example, polyurethane and/or silicone. The conductive traces may be designed to minimize mechanical strain and/or to reduce signal noise, thereby improving the reliability of physiological signal detections made by the ring device.

Flexible substrate 210 may incorporate regions of higher stiffness including, for example, areas to which larger and/or heavier components such as port 240 and/or battery 230 are mounted. Stiffness in such regions may minimize the risk of patch device 200 experiencing any fracture or tearing failures, allowing a ring device releasably attached to patch device 200 to maintain reliable electrical and/or mechanical contact with port 240. Such a region of higher stiffness may thus help to preserve the integrity of the docking interface, ensuring that the ring device remains substantially aligned and/or functional during periods of significant user motion. By reducing elastic deformation, regions of higher stiffness may also help protect embedded electrical components, for example connectors, electrodes, and/or wiring, from deformation-related damage. In some implementations, regions of higher stiffness may include polycarbonate, acrylonitrile butadiene styrene, and/or inserts formed of metal such as stainless steel and/or aluminum. Other regions of flexible substrate 210 may be designed to be softer and/or more flexible, for example formed of the above-mentioned elastomeric base layer, to increase user comfort during an extended monitoring period.

Battery 230 may be a high energy density, lightweight battery, for example a rechargeable lithium-ion, lithium-polymer battery, and/or a disposable single-use button cell battery. As discussed above, battery 230 may be connected to an inductive charger (e.g., including an inductive charging coil 231) to transfer energy to charge a ring device when releasably attached to port 240. Additionally, battery 230 may transfer energy directly to dedicated pulse oximetry sensor 260, ensuring continuous monitoring of the user's blood oxygen levels and/or pulse rate. To optimize performance, battery 230 may include integrated protection circuitry to prevent overcharging and/or thermal runaway. In some implementations, battery 230 may support a capacity sufficient for powering patch device 200 and/or charging an attached ring device for up to two weeks of continuous operation. Furthermore, it may be designed to operate within a wide temperature range and within a variety of environmental conditions. For example, the battery may be waterproof to enable operation in humid environments and/or to allow a limited amount of submerging in water. The battery may be designed to be compliant with industry-standard ingress protection (IP) ratings, for example, IP65. This waterproofing may be achieved by coating battery 230 with a water-resistant coating such as polyurethane and/or silicone, and/or by enclosing battery 230 within a sealed housing made of a water-resistant polymer.

Dedicated pulse oximetry sensor 260 may include an LED and a photodetector configured to measure the user's blood oxygen saturation and/or pulse rate. Pulse oximetry sensor 260 may extend through flexible substrate 210, ensuring direct contact with the user's skin to improve signal quality and/or measurement accuracy. The sensor may be powered by battery 230, enabling continuous operation during extended monitoring periods. In some implementations, pulse oximetry sensor 260 may also include integrated noise-reduction algorithms and/or filters to account for artifacts caused by motion of the user. Pulse oximetry sensor 260 may be housed and/or coated in a protective, water-resistant material to maintain functionality in humid environments and/or withstand exposure to water and/or user perspiration.

As described above, port 240 may enable a ring device to be releasably attached to patch device 200, thereby enabling the ring device to interface to various portions of the flexible substrate. For example, as described above, once releasably attached to port 240, a ring device may be charged using an inductive charger (e.g., including an inductive charging coil 231) that may be embedded within flexible substrate 210 and connected to battery 230, for example via a wiring layer embedded within flexible substrate 210. In some implementations, the inductive charger may include coil 231 integrated into a portion of flexible substrate 210 beneath and/or proximate to port 240. For example, coil 231 of the inductive charger may be positioned to align with a corresponding receiving coil within an attached ring device. This alignment may ensure energy is efficiently transferred from the inductive charger to the ring device during charging. Additionally or alternatively, port 240 may include a dedicated charging connector to interface to a dedicated charging electrode of an attached ring device. Patch device 200 and/or flexible substrate 210 may include one or more markings to ensure a user may align the dedicated charging connector on patch device 200 with the dedicated charging electrode on a ring device.

In some implementations, the inductive charger may be configured to deliver variable levels of energy, allowing it to charge an attached ring device while simultaneously supporting the ring device's operation during continuous physiological monitoring. For example, the inductive charger may operate in both passive and active modes, increasing the energy transferred to an attached ring device during periods of inactivity or reduced monitoring, ensuring uninterrupted functionality of the ring device for extended monitoring periods. In some implementations, the inductive charger may include overcurrent and/or thermal protection circuits to ensure safe operation during prolonged use. Inductive charging may thus enable patch device 200 to support continuous collection of data including, for example electrocardiography, pulse oximetry, and/or acceleration data without disrupting monitoring to recharge a ring device releasably attached to patch device 200.

In addition to enabling an attached ring device to be charged via an inductive charger embedded within flexible substate 210, port 240 may additionally or alternatively include at least one inner connector for interfacing to at least one electrode located at an inward-facing surface of an attached ring device and/or at least one outer connector for interfacing to at least one electrode located at an outward-facing surface of an attached ring device. For example, as shown in FIG. 2A, port 240 may include inner connectors 242a and/or 242b, and/or outer connectors 244a and/or 244b. Inner connectors 242a and/or 242b may electrically couple to one of the electrodes of flexible substrate 210, such as a second electrode, while outer connectors 244a and/or 244b may electrically couple to another electrode of flexible substrate 210, such as a first electrode. These connectors may enable electrodes located at the inner-facing and/or outward-facing surface of an attached ring device to electrically coupled to an electrode of flexible substrate 210 and/or to be securely attached to and/or retained by patch device 200.

To ensure secure electrical coupling and/or mechanical attachment, these connectors may include conical and/or tapered interfaces that guide a ring device into alignment as it is releasably attached to port 240, thereby improving connection reliability. The conical and/or tapered design may allow a ring device to self-center within port 240 during attachment, even if initially misaligned. In some implementations, tapered interfaces on inner and/or outer connectors may include steps to provide a user tactile feedback in the form of an incremental increase in friction as a ring device is attached to port 240. Such steps on tapered interfaces may also accommodate differently sized rings, allowing the same patch device to securely dock ring devices designed for different user finger sizes. For example, a tapered interface may include multiple gradations or layers of tapering, each corresponding to a particular ring device inner and/or outer diameter, or a particular range of inner and/or outer diameters, thereby ensuring that a snug and/or secure attachment may be created for ring devices of different sizes. Such tapered designs thereby ensure a range of different ring devices may be aligned, electrically coupled, and/or mechanically secured to port 240.

Additionally or alternatively, patch device 200 may be offered in two or more versions, with inner and/or outer connectors spaced differently for different versions to accommodate varying ring device sizes. For example, different versions of patch device 200 may include connector spacings and/or connector dimensions designed for specific ranges of ring device inner and/or outer diameters, providing optimized mechanical and/or electrical coupling for ring devices of particular sizes. For example, a “small” version of patch device 200 may include closer-spaced inner and/or outer connectors to enable secure attachment of ring devices of smaller diameter, while a “large” version of patch device 200 may include wider inner and/or outer connector spacings to enable secure attachment of ring devices of larger diameter. For example, different versions of patch device 200 may include differently spaced inner and/or outer connectors that are tapered to enable a precise fit between port 240 and an attached ring device.

To increase the ability of the connectors to hold in place an attached ring device, and/or to protect the surfaces of an attached ring device, tapered interfaces discussed above may include a flexible and/or compressible lining, such as electrically conductive silicone, an electrically conductive elastomeric material, and/or materials embedded with conductive particles. Via the addition of such a lining, inner and/or outer connectors disclosed above may adapt to slight variations in ring device size and/or further increase the electrical and/or mechanical coupling between the inner and/or outer connectors and an attached ring device. Such conductive linings may increase electrical coupling by increasing the surface area of contact between the connector and ring electrode it is contacting, thereby reducing electrical resistance at the point of contact. Such conductive linings may increase mechanical coupling by increasing the friction between surfaces of an attached ring device and the linings as a result of the compressibility and/or coefficient of friction of, for example, an elastomeric lining.

Additionally or alternatively, in some implementations, inner and/or outer connectors may form spring-loaded interfaces. Such spring-loaded interfaces may ensure consistent pressure between electrodes of an attached ring device and inner and/or outer connectors of port 240, thereby reducing electrical resistance and/or increasing reliability of contact formation. Surfaces of such spring-loaded interfaces on inner and/or outer connectors may be flat and/or may be tapered as in the above-described implementation including tapered and/or conical interfaces. One or more such spring-loaded interfaces on inner and/or outer connectors may include a flexible and/or compressible lining that may be electrically conductive as described above.

Additionally or alternatively, surfaces of such spring-loaded interfaces may include a lip at the top portion of the interfaces, for example the portion that would be located the furthest in the foreground relative to FIG. 2A and that a ring device first contacts as a user attaches the ring device to port 240, that may be of a different diameter than the rest of the surface of a particular interface. For example, outer connectors such as outer connector 244a and/or 244b may include a lip at the top portion of the connector that faces inward and is of lower diameter relative to at least the portion of the connector immediately below the lip, thereby capturing and/or holding in place an attached ring device once fully attached to port 240. Additionally or alternatively, inner connectors such as inner connector 242a and/or 242b may include a lip at the top portion of the connector that faces outward and is of higher diameter relative to at least the portion of the connector immediately below the lip, thereby capturing and/or holding in place an attached ring device once fully attached to port 240.

For added retention, port 240 may include a locking feature, such as a latching mechanism, to hold an attached ring device firmly in place once releasably attached to port 240. Such a locking mechanism may engage automatically as a ring device is attached to port 240, providing a secure fit and/or reducing the likelihood that the attached ring device will be accidentally dislodged during user movement and/or prolonged activity. Examples of such locking mechanisms include a spring-loaded latch, a magnetic catch, and/or a flexible strap that ensure the ring device remains aligned and/or mechanically and/or electrically coupled to port 240, while enabling a user to readily remove an attached ring device when desired. For example, a spring-loaded latch may automatically extend a ring device is attached to port 240 and may be manually reset and/or opened to remove the attached ring device.

For example, a magnetic catch may include one or more permanent magnets and/or electromagnets in port 240 and a ring device may enable a magnetic attraction that aligns a ring device to port 240, retains an attached ring device within port 240, and/or holds an attached ring device against the inner and/or outer connectors of port 240. For example, a flexible strap may include an elastic strap attached to port 240, for example to a connector, and/or flexible substrate 210 that a user manually moves over the top of an attached ring device to hold it in place once attached to port 240. Additionally or alternatively, an exemplary flexible strap may be attached to port 240, for example to a connector, and/or flexible substrate 210 on one side and include a snap and/or magnetic clasp on the other to interface to a corresponding snap and/or magnetic clasp on port 240, for example on a connector, and/or flexible substrate 210. In this way, the flexibles strap may be attached over the top of a ring device once attached to port 240 to hold it in place.

To ensure port 240 electrically couples to electrodes at inward-facing and/or outward-facing electrodes of an attached ring device, inner and/or outer connectors of port 240 may be constructed from one or more electrically conductive materials including, for example, copper, brass, aluminum, and/or a conductive polymer. In some implementations, surfaces of inner and/or outer connectors may include a conductive plating and/or coating including, for example, gold and/or nickel, to improve electrical conductivity and/or reduce contact resistance.

Additionally or alternatively, one or more clip connectors may be used to electrically couple one or more of the electrodes of flexible substrate 210 to a ring electrode. For example, a clip connector may include a spring-loaded mechanism and/or a hinged design that securely grips the ring electrode, ensuring consistent electrical contact. The clip connector may also be electrically coupled to an electrode of flexible substrate 210 via conductive traces embedded within the substrate, enabling efficient transmission of electrical signals between the ring electrode and the substrate electrode. These connectors may be constructed from a conductive materials such as copper and may include an insulating coating to prevent unintended electrical contact or damage. Such connectors may be used in place of one or more above-described connectors, for example outer connectors 244a and/or 244b, and/or may add redundancy to electrical couplings, improving the reliability of connections even during user motion.

Additionally or alternatively, for ring devices that include an electrode located at the outward-facing surface that extends to one or more sides of the ring device, patch device 200 may include a base connector that electrically couples to this outer electrode of the ring device. For example, a base connector may be attached to flexible substrate 210 at the base of port 240 and may contact the annular side of the ring device that faces flexible substrate 210 when a ring device is releasably attached to port 240. Similar to above-described connectors, an exemplary base connector may be constructed from one or more electrically conductive materials and may include a flexible and/or compressible lining that may be electrically conductive. An exemplary based connector may be used in place of one or more above-described connectors, for example outer connectors 244a and/or 244b, and/or may add redundancy to electrical couplings, improving the reliability of connections even during user motion.

FIG. 2B depicts a top view of patch device 200 with a ring device 270 releasably attached to port 240 of patch device 200. As shown, inner connectors 242a and/or 242b may interface to one or more inward-facing electrodes of ring device 270, and/or outer connectors 244a and/or 244b may interface to one or more outward-facing electrodes of ring device 270. Inner connectors 242a and/or 242b and/or outer connectors 244a and/or 244b may be configured to electrically and/or mechanically couple to corresponding electrodes of ring device 270, for example via one or more of the above-described design features and/or techniques.

Port 240 may additionally or alternatively include light pipe 250 configured to be located proximate to a pulse oximetry sensor on a ring device, for example pulse oximetry sensor 278 on ring device 270, when said ring device is attached to port 240, as shown in FIGS. 2A and 2B. Such a configuration may enable measurement of blood oxygen saturation (SpO2) of the user and/or pulse rate of the user with or without use of additional sensors or components on patch device 200. Patch device 200 and/or flexible substrate 210 may include one or more markings to ensure a user may align pulse oximetry sensor 278 on ring device 270 with light pipe 250 to ensure a sufficient amount of light is transmitted to and received from a user's skin.

Light pipe 250 may be designed to guide light emitted by an LED of pulse oximetry sensor 278 of ring device 270 to a user's skin and/or return light from the user's skin, for example reflected light, to the photodetector of pulse oximetry sensor 278. Light pipe 250 may be configured to accommodate an orientation of the LED and photodetector of pulse oximetry sensor 278 that is parallel to the surface of flexible substrate 210 following attachment of ring device 270 to port 240. This accommodation may involve redirecting light traveling through light pipe 250 from a propagation direction normal to the surface of flexible substrate 210, for example normal to the surface of a user's skin, to a direction parallel to flexible substrate 210, for example normal to the LED and photodetector of pulse oximetry sensor 278, thereby allowing light emitted by pulse oximetry sensor 278 to reach the user's skin and vice versa.

For example, light pipe 250 may incorporate angled surfaces and/or internal mirrors to redirect light. In some implementations, light pipe 250 may include a 90-degree bend with reflective coatings and/or total internal reflection surfaces to efficiently transmit light from the LED of pulse oximetry sensor 278 to the user's skin and from the user's skin to the photodetector of pulse oximetry sensor 278. The geometry of light pipe 250 may also include optical elements that diffuse and/or collimate light to increase the uniformity of light incident on the user's skin and/or to increase the amount of returned light reaching the photodetector. In some implementations, light pipe 250 may be constructed from optically transparent polymers including, for example, polycarbonate and/or acrylic, and/or designed to minimize scattering of light. Components making up light pipe 250 may include coatings to improve light transmission efficiency and/or prevent ambient light interference.

By utilizing pulse oximetry sensor 278 of ring device 270, patch device 200 may measure both electrocardiography data and pulse oximetry data without involving an additional interface and/or device, simplifying the design and/or power requirements of patch device 200. However, additionally or alternatively using dedicated pulse oximetry sensor 260, optionally included on patch device 200, to measure blood oxygen saturation and/or pulse rate of a user may offer several benefits. For example, pulse oximetry sensor 260 may provide more stable readings by reducing dependence on the alignment of ring device 270 with port 240, for example alignment between pulse oximetry sensor 278 and light pipe 250. This in turn may ensure continuity of blood oxygen saturation and/or pulse rate measurements during movement and/or removal of ring device 270. Furthermore, use of dedicated pulse oximetry sensor 260 may enable simultaneous blood oxygen saturation and/or pulse rate measurement based on signals from both sensor 278 and sensor 260. This in turn may enable validation of measurements from each sensor and/or gaps in measurement data to be filled in the event data from one sensor is temporarily degraded.

Patch device 200 may include an input device, for example a mechanical button or a capacitive touch sensor, that functions as a symptom marker, allowing a user to mark an event and/or an event time for later review by a physician. Activation of this input device may cause patch device 200 to transmit information to a controller and/or memory of patch device 200, and/or to ring device 270. The marked event may be transferred from patch device 200 and/or from ring device 270 to an external device such as a mobile phone or other app-enabled device. Upon receiving the marked event or events, the mobile phone and/or app may prompt the user to input, for example by dictation, additional details about the event and/or symptoms that the user experienced at the time of activation of the input device. For example, the user may provide contextual information, such as chest pain, dizziness, or shortness of breath, through text entry and/or voice recognition. This information may then be associated with physiological data recorded by patch device 200 and/or ring device 270 during the period before and/or after activation of the input device, enabling physician review of relevant data.

In some implementations, the input device may include tactile and/or visual feedback, for example a mechanical button click and/or a change in a LED indicator, to confirm receipt of the input. Additionally, to prevent accidental user input, the input device may be recessed and/or have a locking capability. A marked event may be associated with a timestamp to enable synchronization with physiological data measured by patch device 200, and/or ring device 270.

Patch device 200 and/or ring device 270 may include an LED indicator light, speaker, and/or vibration motor to alert a user and/or caregiver following detection of one or more events including, for example, a sleep apnea and/or respiratory distress event and/or a detection that a user has fallen. An LED indicator light of patch device 200 and/or ring device 270 may emit a visible signal, such as a flashing or steady light of a specific color, to provide a visual alert. For example, a red LED may indicate a critical health event, such as respiratory distress or a fall, while a different color, such as green, may indicate normal operation or successful system connection. The LED indicator may be located on an easily visible area of patch device 200 and/or ring device 270, ensuring the user and/or caregiver may quickly become aware of the alert.

A speaker of patch device 200 and/or ring device 270 may emit an auditory signal including, for example, a beep and/or spoken alert. The speaker may be programmed to use a different tone and/or volume to distinguish between various events. For example, a continuous beep may indicate a critical event, such as a fall, while an intermittent tone may indicate a less urgent issue, such as a low battery warning.

A vibration motor of patch device 200 and/or ring device 270 may generate a tactile alert including, for example, a short or long vibration pattern. The vibration motor may be programmed to use different durations and/or intensities to distinguish between various events. For instance, a continuous vibration may indicate a critical event, such as a fall or respiratory distress, while a series of brief pulses may indicate a less urgent issue, such as a low battery warning. Events that trigger such visual, auditory, and/or tactile alerts may additionally or alternatively trigger notifications on an external computing device communicatively coupled to patch device 200 and/or ring device 270, for example a mobile phone.

FIG. 2C depicts a detail view of port 240 of patch device 200 with ring device 270 attached to port 240. As described above, port 240 may include inner connectors 242a and/or 242b, which may be configured to electrically couple to two electrodes 272a and 272b located at the inward-facing surface of ring device 270. Inner connectors 242a and/or 242b may in turn be electrically coupled to an electrode of patch device 200, for example a second electrode. As shown, port 240 may also include outer connectors 244a and/or 244b, which may be configured to electrically couple to an electrode 274 located at the outward-facing surface of ring device 270. Outer connectors 244a and/or 244b may in turn be electrically coupled to an electrode of patch device 200, for example a first electrode.

Instead of a single electrode located at the inward-facing surface of ring device 270 and a single electrode located at outward-facing surface of ring device 270, ring device 270 may instead include two or more electrodes located at the inward-facing surface of ring device 270 and/or two or more electrodes located at the outward-facing surface of ring device. In such an implementation, port 240 may include a corresponding number of inner and/or outer connectors to ensure port 240 may electrically couple to some or all of the electrodes on ring device 270. Patch device 200 and/or flexible substrate 210 may include one or more markings to ensure a user may align the electrodes of ring device 270 with corresponding inner and/or outer connectors to ensure a sufficient electrical and/or mechanical coupling is established.

In some implementations in which ring device 270 includes two or more inner electrodes and/or two or more outer electrodes, two electrodes of patch device 200, for example a first electrode and a second electrode, may each be electrically coupled via an inner connector to a separate inner electrode of ring device 270. In other implementations in which ring device 270 includes two or more inner electrodes and/or two or more outer electrodes, two electrodes of patch device 200 may each be electrically coupled via an outer connector to a separate outer electrode of ring device 270. In other implementations in which ring device 270 includes two or more inner electrodes and/or two or more outer electrodes, one electrode of patch 200 may be electrically coupled via an inner connector to an inner electrode of ring device 270 and one electrode of patch 200 may be electrically coupled via an outer connector to an outer electrode of ring device 270.

Patch device 200 may include a plurality of electrodes configured to interface to a plurality of outer electrodes of ring device 270. For example, patch device 200 may include at least two electrodes, at least three electrodes, at least four electrodes, at least five electrodes, or at least six electrodes. Patch device may include at most six electrodes, at most five electrodes, at most four electrodes, at most three electrodes, or at most two electrodes. Each of the plurality of electrodes of patch device 200 may be positioned at a distinct recording location on flexible substrate 210. Each of the plurality of electrodes of patch device 200 may be routed to a corresponding plurality of outer connectors of port 240. For example, port 240 may include at least two outer connectors, at least three outer connectors, at least four outer connectors, at least five outer connectors, at least six outer connectors, at most six outer connectors, at most five outer connectors, at most four outer connectors, at most three outer connectors, or at most two outer connectors. Each outer connector may be configured to electrically couple to a corresponding outer electrode of ring device 270. For example, ring device 270 may include at least two outer electrodes, at least three outer electrodes, at least four outer electrodes, at least five outer electrodes, at least six outer electrodes, at most six outer electrodes, at most five outer electrodes, at most four outer electrodes, at most three outer electrodes, or at most two outer electrodes.

The plurality of electrodes of patch device 200 may be routed to different outer electrodes of ring device 270. Patch device 200 and ring device 270 may thus enable measurements (e.g., simultaneous measurements) of difference potentials between multiple pairs of the plurality of electrodes positioned at distinct recording locations on flexible substrate 210. For example, the plurality of electrodes of patch device 200 may be positioned and configured to form a precordial lead set. These measurements may collectively be used to generate a plurality of cardiac electrical activity signals corresponding to a multi-lead electrocardiogram and/or a derived vectorcardiogram. For example, the plurality of electrodes of patch device 200 may be arranged across the chest region of a user to record distinct cardiac electrical activity signals, and ring device 270 may sequentially or simultaneously acquire difference potentials between electrodes of patch device 200 to reconstruct independent ECG leads (e.g., to reconstruct six independent ECG leads). Such implementations may enable patch device 200 and ring device 270 to provide multi-lead cardiac monitoring corresponding to a higher spatial resolution relative to a single-lead implementation.

FIG. 2D depicts a side view of patch device 200 with ring device 270 releasably attached to port 240, and FIG. 2E depicts a bottom view of patch device 200. As shown in FIGS. 2D and 2E, light pipe 250 and/or pulse oximetry sensor 260, for example an LED and photodetector of pulse oximetry sensor 260, may be configured to be proximate to a user's skin when patch device 200 is adhered to the user's skin. This may enable light from the LED of pulse oximetry sensor 278 to reach the user's skin via light pipe 250 and/or light from the user's skin to reach the photodetector of pulse oximetry sensor 278 as described above. This may further enable light from the LED of pulse oximetry sensor 260 to reach the user's skin and/or light from the user's skin to reach the photodetector of pulse oximetry sensor 260.

The bottom surface of flexible substrate 210, for example the surface facing the user's skin, may additionally include two or more electrodes, for example a first electrode 212 and/or a second electrode 214, as described above and as shown in FIGS. 2D and 2E. First electrode 212 and/or second electrode 214 may be embedded within and/or attached to flexible substrate 210. As described above, first electrode 212 and/or second electrode 214 may be electrically coupled to port 240 via a wiring layer of flexible substrate 210. For example, first electrode 212 may be electrically coupled to outer connectors 244a and/or 244b of port 240, and second electrode 214 may be electrically coupled to inner connectors 242a and/or 242b of port 240. This may allow ring device 270 to measure the electric potential between first electrode 212 and second electrode 214, generating data to simulate an electrocardiogram.

First electrode 212 and second electrode 214 may be positioned on flexible substrate 210 so as to maximize the distance between the two electrodes. Increasing the distance between electrodes used for electrocardiography may enhance the ability to detect cardiac electrical signals by increasing the potential difference measured between them. This greater separation may allow the electrodes to capture a broader range of cardiac electrical activity and may result in a higher signal-to-noise ratio. The spacing between electrodes may be, for example, at least 1 inch, at least 2 inches, at least 3 inches, at least 4 inches, at least 5 inches, at least 6 inches, at least 7 inches, at least 8 inches, at least 9 inches, at least 10 inches, at most 10 inches, at most 9 inches, at most 8 inches, at most 7 inches, at most 6 inches, at most 5 inches, at most 4 inches, at most 3 inches, at most 2 inches, and/or at most 1 inch. The diameter of first electrode 212 and/or second electrode 214 may be at least 0.25 inches, at least 0.5 inches, at least 0.75 inches, at least 1 inch, at least 1.25 inches, at least 1.5 inches, at least 1.75 inches, at least 2 inches, at most 2 inches, at most 1.75 inches, at most 1.5 inches, at most 1.25 inches, at most 1 inch, at most 0.75 inches, at most 0.5 inches, and/or aat most 0.25 inches.

First electrode 212 and/or second electrode 214 may include conductive gel to improve electrode-skin contact and/or electrical signal quality. In some implementations, first electrode 212 and/or second electrode 214 may be gel electrodes that include an adhesive conductive gel to both adhere patch device 200 to the user's skin and form a conformal interface between one or both electrodes and the user's skin. This conformal interface may in turn improve electrical coupling by minimizing contact resistance, thereby improving electrical signal quality even during user movement. The adhesive conductive gel may also provide a cushioning effect, enhancing user comfort during extended wear. For example, adhesive conductive gel may include silver/silver chloride, hydrogel, polyacrylamide, and/or silicone.

Additionally or alternatively, the bottom surface of flexible substrate 210 may include an adhesive layer. In some implementations, the adhesive layer may include areas of the bottom surface of flexible substrate 210 that surround first electrode 212 and/or second electrode 214. In other implementations, the adhesive layer may include only peripheral regions of the bottom surface of flexible substrate 210. In other implementations, the adhesive layer may include all areas of the bottom surface of flexible substrate 210 apart from first electrode 212 and/or second electrode 214, light pipe 250, and/or pulse oximetry sensor 260. Such an adhesive layer may be used in addition to adhesive gel electrodes, for example first electrode 212 and/or second electrode 214 that include an adhesive conductive gel, or as an alternative to adhesive conductive gel electrodes, for example first electrode 212 and/or second electrode 214 that include a non-adhesive conductive gel.

In other implementations, for example for users with skin sensitivity or irritation, first electrode 212 and/or second electrode 214 may include a dry-contact electrode. Additionally, the adhesive layer of the bottom surface of flexible substrate 210 may include a non-irritating lower tack adhesive.

Such an adhesive layer may ensure secure attachment of patch device 200 to the user's skin, which may in turn prevent dislodgement of the patch device and/or improve the contact made between first electrode 212 and/or second electrode 214 and the user's skin despite user motion. The adhesive layer may include a pressure-sensitive adhesive that is skin-friendly and/or that may allow removal and reapplication, for example an adhesive that includes silicone, acrylic, hydrocolloid, and/or polyurethane.

FIG. 2F depicts a top view of patch device 200 including one or more traces within a wiring layer. For example, as discussed above, wiring traces may include trace 290 connecting outer connectors 244a and/or 244b to first electrode 212, trace 292 connecting inner connectors 242a and/or 242b to second electrode 214, and/or trace 294 connecting battery 230 to pulse oximetry sensor 260. Traces within the wiring layer may additionally or alternatively connect battery 230 to an inductive charger (e.g., including an inductive charging coil 231) charging a ring device when attached to the port 240, as shown in FIGS. 2A and 2B.

Patch device 200 may be configured to withstand exposure to a variety of environmental conditions and/or motion profiles including humidity, water exposure (e.g. during showering and/or swimming), sweat exposure and/or sharp accelerations (e.g. during exercise), and/or static pressure (e.g. while lying in a prone position). To ensure patch device 200 maintains functionality during prolonged exposure to such conditions, several design features may be implemented. The adhesive conductive gel that may be a part of first electrode 212 and/or second electrode 214 may be water-resistant to maintain conductivity and/or adhesion during exposure to sweat and/or water. The adhesive layer securing patch device 200 to the user's skin may include waterproof or water-resistant materials, such as silicone or polyurethane adhesives, to ensure stable attachment in high-moisture environments.

The top side of patch device 200, including the top side of flexible substrate 210, may be coated with protective layers, such as polyurethane and/or silicone, providing a moisture-resistant outer barrier to shield internal components from sweat, water, and/or dust. Additionally or alternatively, some or all of the top side of patch device 200 may be enclosed in a rigid or semi-rigid protective casing, offering additional protection against water ingress and/or mechanical impacts, for example mechanical impacts that may damage electrical components and/or dislodge attached ring device 270. Such a protective casing may include hydrophobic vents and/or seals to allow airflow while maintaining water-resistance.

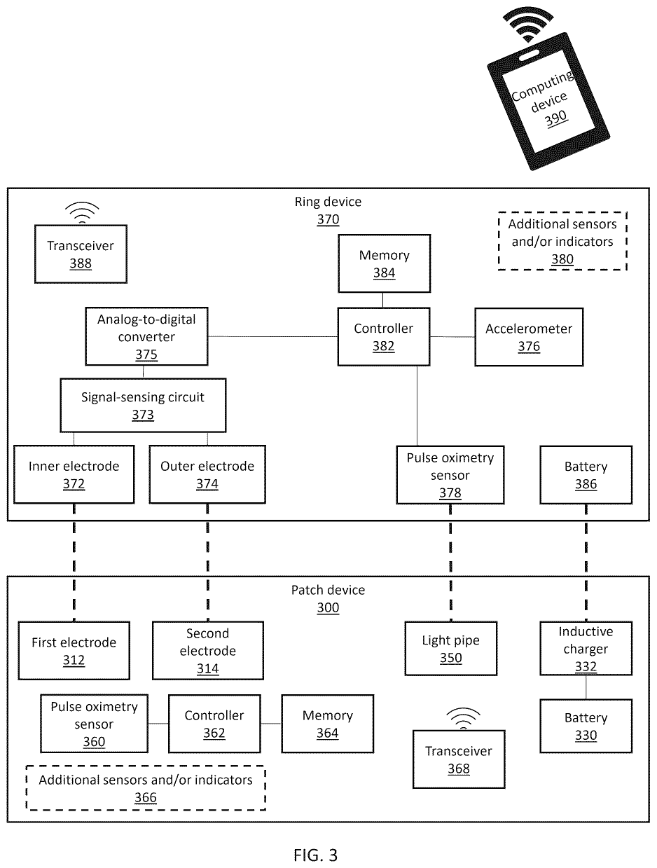

FIG. 3 depicts an exemplary block diagram of components of a ring device 370 and a patch device 300. Ring device 370 may include inner electrode 372 and outer electrode 374, and may be configured to measure the electric potential between the two electrodes. Inner electrode 372 may interface to second electrode 314 of patch device 300 via at least one inner connector of the port of patch device 300, while outer electrode 374 may interface to first electrode 312 of patch device 300 via at least one outer connector of the port of patch device 300. The electric potential measured between inner electrode 372 and outer electrode 374 may be processed by a signal-sensing circuit 373, which may amplify or filter the signal before providing it to an analog-to-digital converter 375. Analog-to-digital converter 375 may digitize the signal and transmit it to controller 382, which may further process the data to generate an electrocardiogram or perform other analyses. Controller 382 may store data in memory 384 and may also interface to additional sensors and/or indicators 380 that may include, for example, one or more gyroscopes, infrared sensors, and/or speakers, vibration motors, and/or LED lights used to generate alarms and/or alerts for users and/or caretakers.

Ring device 370 may also include a pulse oximetry sensor 378 configured to measure a user's blood oxygen saturation and/or pulse rate. Pulse oximetry sensor 378 may include an LED and a photodetector, with light directed to and from the user's skin via light pipe 350 of patch device 300. Controller 382 may process signals from pulse oximetry sensor 378, to measure a user's blood oxygen saturation and/or pulse rate, and/or signals from accelerometer 376, to monitor a user's motion, respiratory patterns, and/or sleep-related metrics.

Ring device 370 may include a transceiver 388 configured to send and/or receive data to and/or from patch device 300 and/or a computing device 390 communicatively coupled to ring device 370 and/or patch device 300. Computing device 390 may take the form of the user's mobile phone, tablet, and/or personal computer, for example. Transmitted data types may include raw physiological signal data (e.g. electrocardiography and/or pulse oximetry data), status updates from ring device 370 and/or patch device 300 (e.g. alerts and/or notifications for the user). In this way, physiological signal data processing may occur on ring device 370 and/or on computing device 390. Additionally, ring device 370 may include a battery 386 that may be, when ring device 370 is removably attached to the port of patch device 300, inductively charged via an inductive charger 332 embedded within patch device 300. Inductive charger 332 may be electrically coupled to battery 330 of patch device 300, enabling consistent power delivery to ring device 370 when ring device 370 is removably attached to the port of patch device 300, thereby ensuring continuous operation during extended monitoring periods.

Patch device 300 may also include a dedicated pulse oximetry sensor 360, which may be connected to a controller 362 and a memory 364 to process and store, respectively, data from sensors including pulse oximetry sensor 360. Controller 362 may also interface to additional sensors and/or indicators 366 that may include, for example, one or more accelerometers, gyroscopes, infrared sensors, symptom marker input devices (e.g. mechanical buttons or capacitive touch sensors), and/or speakers, vibration motors, and/or LED lights used to generate alarms and/or alerts for users and/or caretakers. Data from sensors including pulse oximetry sensor 360 may be transmitted via a transceiver 368 of patch device 300 to transceiver 388 of ring device 370 and/or directly to computing device 390, providing redundancy for and/or validation of data from pulse oximetry sensor 378 of ring device 370.

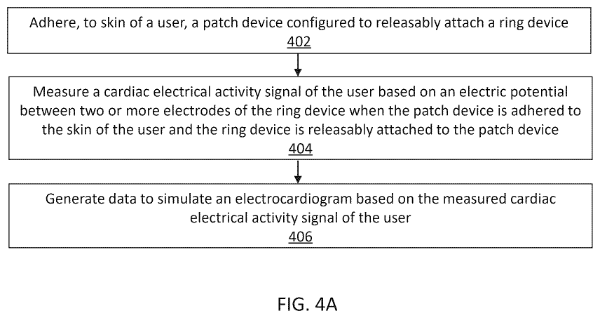

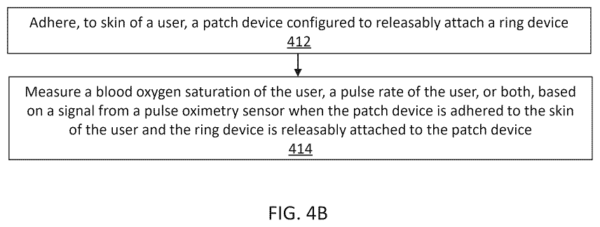

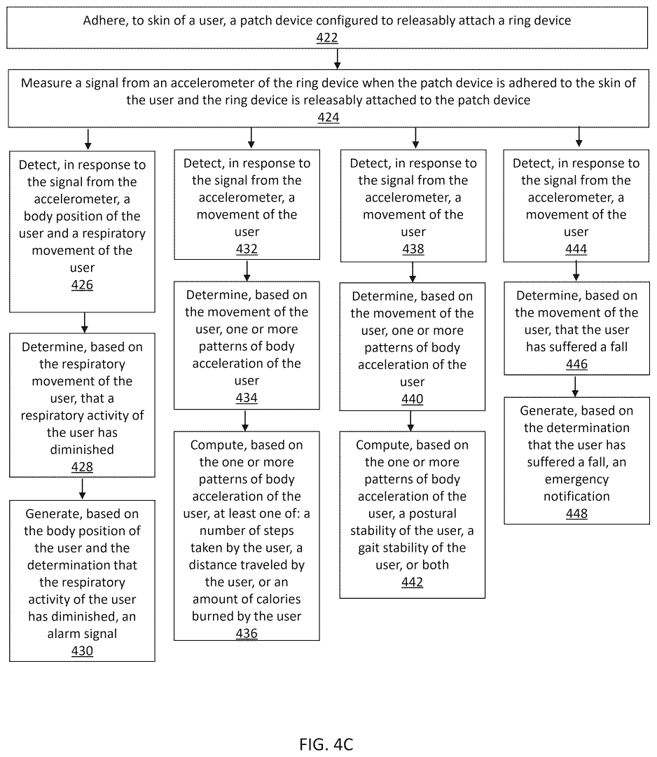

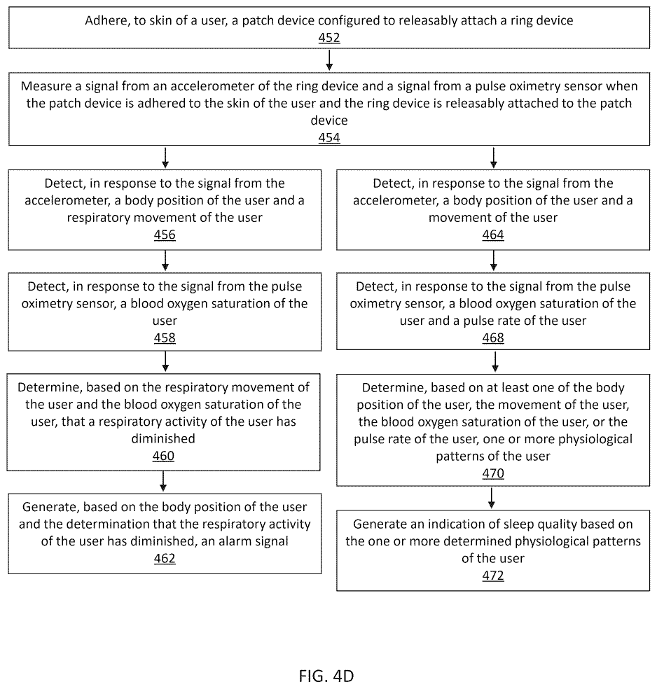

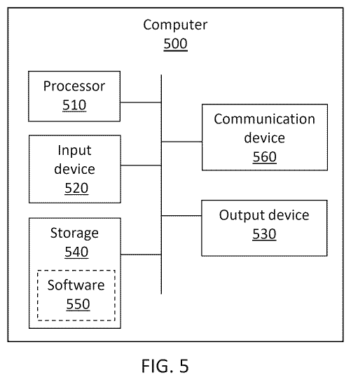

FIG. 4A depicts an exemplary process for cardiac electrical activity monitoring using a patch device and a ring device. At step 402, an exemplary patch device may be adhered to the user's skin, for example at their chest region, ensuring secure placement for prolonged monitoring. The patch device may include a flexible substrate with integrated electrodes and a port configured to releasably attach a ring device. Attaching the ring device to the patch device may allow for electrical and/or mechanical coupling between electrodes on the ring device and connectors on the patch device. At step 404, the ring device may measure a cardiac electrical activity signal by detecting the electric potential across two or more electrodes of the ring device. These electrodes may be electrically coupled to corresponding electrodes on the patch device, which may be positioned for signal acquisition by spanning across the user's heart. At step 406, the ring device may process the measured cardiac electrical activity signal to generate data simulating an electrocardiogram. This simulation may provide a continuous representation of the user's cardiac electrical activity, which may be transmitted to a computing device for further analysis, visualization, and/or storage.

FIG. 4B depicts an exemplary process for monitoring blood oxygen saturation and/or pulse rate of a user using a patch device and a ring device. At step 412, an exemplary patch device may be adhered to the user's skin, for example at their chest region, ensuring secure placement for reliable signal acquisition. The patch device may include a flexible substrate with integrated components such as electrodes, a light pipe, and a port configured to releasably attach a ring device. Attaching the ring device to the patch device may establish electrical, mechanical, and/or optical coupling between the two devices. At step 414, the ring device may measure the user's blood oxygen saturation, pulse rate, or both, using a pulse oximetry sensor integrated into the ring device. This sensor may emit light via an LED and detect light from a user's skin via a photodetector, with light guided to and from the user's skin through a light pipe in the patch device. The signals generated by the pulse oximetry sensor may be processed by a controller on the ring device to calculate physiological parameters such as blood oxygen saturation and/or pulse rate of the user. These measurements may be transmitted to a computing device, such as a mobile phone or tablet, for visualization, analysis, and/or storage.

FIG. 4C depicts an exemplary process for utilizing accelerometer signals from a ring device to monitor and/or analyze various physiological and/or activity-related parameters of a user. At step 422, an exemplary patch device may be adhered to the user's skin, for example at their chest region, ensuring secure placement for prolonged monitoring. The patch device may include a port configured to releasably attach a ring device, establishing electrical and/or mechanical coupling between the two devices. Once the ring device is attached, at step 424, a signal from one or more movement sensors, for example one or more three-axis accelerometers, integrated into the ring device may be measured. This signal may provide data about the user's movements and/or body position, enabling further analysis through one or more of the below processes.

For example, a first process may involve respiratory monitoring (e.g. sleep apnea event detection), and/or alarm generation. At step 426, the ring device may detect the user's body position and/or respiratory movements, for example movement of the user's chest wall, in response to the accelerometer signal. For example, the presence or absence of rhythmic movement be analyzed to determine respiratory activity (whether the user is breathing, the breathing rate, whether breathing has diminished or increased, etc.). The one or more movement sensors may provide information that can be used by the ring device to determine whether the user is lying on their back, lying on their right side, or lying on their left side. At step 428, the ring device may determine, based on a determined reduction in detected respiratory movements, that the user's respiratory activity has diminished, potentially indicating a sleep apnea and/or respiratory distress event. Additionally or alternatively the determination that the user's respiratory activity has diminished may be based on electrocardiography data, for example detection of heart rate variability as indicative of a sleep apnea and/or respiratory distress event. At step 430, an alarm signal may be generated based on the detected body position (e.g. corresponding to lying on the back, lying on the right side, or lying on the left side) and diminished respiratory activity, and may notify the user and/or a caregiver for immediate intervention. For example, the alarm signal may prompt the user to put on a Continuous Positive Airway Pressure (CPAP) device. The alarm may be generated and/or produced at one or more of the ring device, the patch device, and/or a computing device, for example a mobile phone or tablet in communication with the ring device and/or patch device. Use of the patch device to monitor respiratory movements and/or blood oxygen saturation may additionally allow physician assessment of the effectivity of use of the CPAP device.

For example, a second process may involve activity monitoring. At step 432, the ring device may detect the user's movement based on the accelerometer signal. At step 434, the ring device may analyze the detected movement to determine one or more patterns of body acceleration, such as repetitive walking or running motions. At step 436, these patterns may be used to compute activity-related metrics, such as the number of steps taken by the user, the distance traveled by the user, and/or the amount of calories burned by the user. This process may enable fitness tracking and activity monitoring, supporting a user's health and wellness goals.

For example, a third process may involve postural and/or gait stability analysis. At step 438, the ring device may detect the user's movement in response to the accelerometer signal. At step 440, the system may analyze the movement to determine one or more patterns of body acceleration that may provide insights into postural stability, gait stability, or both. Accelerometer data may be combined with pulse oximetry data to further analyze postural stability and/or gait stability. For example, a decrease in a user's pulse rate in combination with unsteady movements and/or stumbling may indicate a bradycardic episode, which in some cases may contribute to reduced postural stability. At step 442, the system may compute metrics related to the user's balance and/or gait, which may be useful for identifying risks of instability or mobility impairments, such as those associated with aging or neurological conditions.

For example, a fourth process may involve fall detection and/or emergency notification. At step 444, the ring device may detect movement of the user based on the accelerometer signal. At step 446, the system may determine, based on the detected movement, that the user has suffered a fall. This determination may involve recognizing sudden deceleration and/or impact patterns characteristic of falls. At step 448, the ring device may generate an emergency notification based on the fall detection. This notification may include details about the incident and/or may be sent to a caregiver, emergency contact, and/or emergency response system to ensure timely assistance.

FIG. 4D depicts an exemplary process for utilizing accelerometer and pulse oximetry sensor data from a ring device to monitor and/or analyze a user's respiratory activity and/or sleep quality. At step 452, an exemplary patch device may be adhered to the user's skin, for example at their chest region, ensuring secure placement for prolonged monitoring. The patch device may include a port configured to releasably attach a ring device, establishing electrical, mechanical, and/or optical coupling between the two devices. Once the ring device is attached, at step 454, signals from both an accelerometer and a pulse oximetry sensor integrated into the ring device may be measured. The accelerometer signal may provide information on the user's movements and/or body position, while the pulse oximetry sensor signal may measure physiological parameters such as a user's blood oxygen saturation (SpO2) and/or pulse rate. These signals may enable advanced monitoring and/or analysis through one or more of the below processes.

For example, a first process may involve respiratory monitoring (e.g. sleep apnea event detection), and/or alarm generation. At step 456, the ring device may detect the user's body position and/or respiratory movements in response to the accelerometer signal. For example, the presence or absence of rhythmic changes in chest position may indicate whether respiratory activity is present or has diminished while patterns in the signal may indicate whether the user is lying on their back, lying on their right side, or lying on their left side. At step 458, the ring device may use the pulse oximetry signal to detect the user's blood oxygen saturation. At step 460, the system may determine that the user's respiratory activity has diminished based on a combination of respiratory movement data from the accelerometer and blood oxygen saturation data from the pulse oximetry sensor. For example, this determination may be based on a reduction in blood oxygen saturation and/or respiratory movement and may indicate the user is experiencing a sleep apnea and/or respiratory distress event. Additionally or alternatively the determination that the user's respiratory activity has diminished may be based on electrocardiography data, for example detection of heart rate variability as indicative of a sleep apnea and/or respiratory distress event. At step 462, an alarm signal may be generated based on the detected body position (e.g. corresponding to lying on the back, lying on the right side, or lying on the left side) and the determination that respiratory activity has diminished. This alarm signal may notify the user and/or a caregiver for immediate intervention. For example, the alarm signal may prompt the user to put on a Continuous Positive Airway Pressure device. The alarm may be generated and/or produced at one or more of the ring device, the patch device, and/or a computing device, for example a mobile phone or tablet in communication with the ring device and/or patch device.

For example, a second process may involve sleep quality analysis. At step 464, the ring device may detect the user's body position and/or movement using the accelerometer signal. For example, patterns in the signal may identify whether the user is lying prone, supine, or on their side, as well as capture transitions between these positions. At step 468, the ring device may measure the user's blood oxygen saturation and/or pulse rate using the pulse oximetry sensor signal. At step 470, the system may analyze these inputs including, for example, body position and/or angle, movement, blood oxygen saturation, and/or pulse rate, to identify one or more physiological patterns of the user, such as respiratory patterns, movement frequency, and/or heart rate variability during sleep. At step 472, these determined physiological patterns may be used to generate an indication of the user's sleep quality. This sleep quality assessment may include metrics such as sleep stages, time spent in deep sleep, and/or number of interruptions caused by respiratory or movement disturbances, allowing a user to monitor and/or improve their sleep health.

In one or more examples, the disclosed systems and methods utilize or may include a computer system. FIG. 5 depicts an exemplary computing system according to one or more examples of the disclosure. Computer 500 can be a host computer connected to a network. Computer 500 can be a client computer or a server. As shown in FIG. 5, computer 500 can be any suitable type of microprocessor-based device, such as a personal computer, workstation, server, or handheld computing device, such as a phone or tablet. The computer can include, for example, one or more of processor 510, input device 520, output device 530, storage 540, and communication device 560. Input device 520 and output device 530 can correspond to those described above and can either be connectable or integrated with the computer.

Input device 520 can be any suitable device that provides input, such as a touch screen or monitor, keyboard, mouse, or voice-recognition device. Output device 530 can be any suitable device that provides an output, such as a touch screen, monitor, printer, disk drive, or speaker.

Storage 540 can be any suitable device that provides storage, such as an electrical, magnetic, or optical memory, including a random-access memory (RAM), cache, hard drive, CD-ROM drive, tape drive, or removable storage disk. Communication device 560 can include any suitable device capable of transmitting and receiving signals over a network, such as a network interface chip or card. The components of the computer can be connected in any suitable manner, such as via a physical bus or wirelessly. Storage 540 can be a non-transitory computer-readable storage medium comprising one or more programs, which, when executed by one or more processors, such as processor 510, cause the one or more processors to execute methods described herein.

Software 550, which can be stored in storage 540 and executed by processor 510, can include, for example, the programming that embodies the functionality of the present disclosure (e.g., as embodied in the systems, computers, servers, and/or devices as described above). In one or more examples, software 550 can include a combination of servers such as application servers and database servers.

Software 550 can also be stored and/or transported within any computer-readable storage medium for use by or in connection with an instruction execution system, apparatus, or device, such as those detailed above, that can fetch and execute instructions associated with the software from the instruction execution system, apparatus, or device. In the context of this disclosure, a computer-readable storage medium can be any medium, such as storage 740, that can contain or store programming for use by or in connection with an instruction execution system, apparatus, or device.

Software 550 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include but is not limited to, an electronic, magnetic, optical, electromagnetic, or infrared wired or wireless propagation medium.

Computer 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer 500 can implement any operating system suitable for operating on the network. Software 550 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments and/or examples. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A patch device for monitoring cardiac electrical activity, the patch device comprising:
a flexible substrate configured to adhere to skin of a user and comprising two or more electrodes; and
a port for releasably attaching a ring device, wherein the ring device is configured to be worn around a finger of the user and comprises a monitor configured to monitor cardiac electrical activity of the user, wherein the port is configured to electrically couple the two or more electrodes of the flexible substrate to the monitor of the ring device.

2. The patch device of claim 1, wherein the two or more electrodes of the flexible substrate comprise electrically conductive adhesive gel configured to adhere to the skin of the user.

3. The patch device of claim 1, wherein the at least one cardiac electrical activity signal is based on an electric potential between the two or more electrodes of the flexible substrate.

4. The patch device of claim 1, wherein the ring device comprises two or more electrodes configured to detect cardiac electrical activity when contacting the skin of the user.

5. The patch device of claim 4, wherein the port is configured electrically couple at least one of the two or more electrodes of the flexible substrate to at least one electrode of the two or more electrodes of the ring device when the ring device is releasably attached to the port.

6. The patch device of claim 5, wherein:
the port comprises at least one inner connector, at least one outer connector, or both; and
at least one connector, selected from a group consisting of the at least one inner connector and the at least one outer connector, comprises a tapered interface, a spring-loaded interface, or a tapered spring-loaded interface.

7. The patch device of claim 6, wherein the port comprises:
at least one inner connector configured to electrically couple at least one electrode located at an inward-facing surface of the ring device to a first electrode of the two or more electrodes of the flexible substrate; and
at least one outer connector configured to electrically couple at least one electrode located at an outward-facing surface of the ring device to a second electrode of the two or more electrodes of the flexible substrate.

8. The patch device of claim 6, wherein the port comprises:
a first inner connector configured to electrically couple a first electrode located at an inward-facing surface of the ring device to a first electrode of the two or more electrodes of the flexible substrate; and
a second inner connector configured to electrically couple a second electrode located at an inward-facing surface of the ring device to a second electrode of the two or more electrodes of the flexible substrate.

9. The patch device of claim 6, wherein the port comprises:
a first outer connector configured to electrically couple a first electrode located at an outward-facing surface of the ring device to a first electrode of the two or more electrodes of the flexible substrate; and
a second outer connector configured to electrically couple a second electrode located at an outer-facing surface of the ring device to a second electrode of the two or more electrodes of the flexible substrate.

10. The patch device of claim 1, further comprising a light pipe, wherein the light pipe is configured to direct light between the skin of a user and a pulse oximetry sensor of the ring device.

11. The patch device of claim 1, wherein the patch device further comprises a pulse oximetry sensor configured to measure a blood oxygen saturation of the user, a pulse rate of the user, or both, wherein the pulse oximetry sensor comprises an LED and a photodetector.

12. The patch device of claim 1, wherein the patch device further comprises a transceiver configured to transmit data from the pulse oximetry sensor to a transceiver of the ring device.

13. The patch device of claim 1, further comprising a battery for charging the ring device.

14. The patch device of claim 1, wherein the patch device is configured to inductively charge the ring device.

15. An apparatus for monitoring cardiac electrical activity, the apparatus comprising:
a ring device configured to be worn around a finger of a user and comprising a monitor configured to monitor the cardiac electrical activity of the user;
a flexible substrate configured to adhere to skin of the user and comprising two or more electrodes; and
a port configured to releasably attach the ring device and electrically couple the two or more electrodes of the flexible substrate to the monitor of the ring device.

16. A method for monitoring a user, the method comprising:
adhering, to skin of a user, a patch device configured to releasably attach a ring device;
measuring a cardiac electrical activity signal of the user based on an electric potential between two or more electrodes of the ring device when the patch device is adhered to the skin of the user and the ring device is releasably attached to the patch device; and
generating data to simulate an electrocardiogram based on the measured cardiac electrical activity signal of the user.

17. A method for monitoring a user, the method comprising:
adhering, to skin of a user, a patch device configured to releasably attach a ring device; and
measuring a blood oxygen saturation of the user, a pulse rate of the user, or both, based on a signal from a pulse oximetry sensor when the patch device is adhered to the skin of the user and the ring device is releasably attached to the patch device.

18. A method for monitoring a user, the method comprising:
adhering, to skin of a user, a patch device configured to releasably attach a ring device; and
measuring a signal from an accelerometer of the ring device when the patch device is adhered to the skin of the user and the ring device is releasably attached to the patch device.

19. The method of claim 18, the method further comprising:
detecting, in response to the signal from the accelerometer, a body position of the user and a respiratory movement of the user;
determining, based on the respiratory movement of the user, that a respiratory activity of the user has diminished; and
generating, based on the body position of the user and the determination that the respiratory activity of the user has diminished, an alarm signal.

20. The method of claim 19, wherein the alarm signal is generated based on a detection of at least one body position of the user selected from a group comprising: lying on the back, lying on the right side, and lying on the left side.

21. The method of claim 18, the method further comprising:
detecting, in response to the signal from the accelerometer, a movement of the user;
determining, based on the movement of the user, one or more patterns of body acceleration of the user; and
computing, based on the one or more patterns of body acceleration of the user, at least one of: a number of steps taken by the user, a distance traveled by the user, or an amount of calories burned by the user.

22. The method of claim 18, the method further comprising:
detecting, in response to the signal from the accelerometer, and a movement of the user;
determining, based on the movement of the user, one or more patterns of body acceleration of the user; and
computing, based on the one or more patterns of body acceleration of the user, a postural stability of the user, a gait stability of the user, or both.

23. The method of claim 18, the method further comprising:
detecting, in response to the signal from the accelerometer, a movement of the user;
determining, based on the movement of the user, that the user has suffered a fall; and
generating, based on the determination that the user has suffered a fall, an emergency notification.

24. A method for monitoring a user, the method comprising:
adhering, to skin of a user, a patch device configured to releasably attach a ring device; and
measuring a signal from an accelerometer of the ring device and a signal from a pulse oximetry sensor when the patch device is adhered to the skin of the user and the ring device is releasably attached to the patch device.

25. The method of claim 24, the method further comprising:
detecting, in response to the signal from the accelerometer, a body position of the user and a respiratory movement of the user;
detecting, in response to the signal from the pulse oximetry sensor, a blood oxygen saturation of the user;
determining, based on the respiratory movement of the user and the blood oxygen saturation of the user, that a respiratory activity of the user has diminished; and
generating, based on the body position of the user and the determination that the respiratory activity of the user has diminished, an alarm signal.

26. The method of claim 25, wherein the alarm signal is generated based on a detection of at least one body position of the user selected from a group comprising: lying on the back, lying on the right side, and lying on the left side.

27. The method of claim 24, the method further comprising:
detecting, in response to the signal from the accelerometer, a body position of the user and a movement of the user;
detecting, in response to the signal from the pulse oximetry sensor, a blood oxygen saturation of the user and a pulse rate of the user;
determining, based on at least one of the body position of the user, the movement of the user, the blood oxygen saturation of the user, or the pulse rate of the user, one or more physiological patterns of the user; and
generating an indication of sleep quality based on the one or more determined physiological patterns of the user.
